(19) United States
(12) Patent Application Publication
(10) Pub. No.: US 2025/0144420 A1
(43) Pub. Date: May 8, 2025

(54) HEADPIECE CONFIGURATIONS OF A COCHLEAR IMPLANT SYSTEM

(71) Applicant: Advanced Bionics AG, Staefa (CH)

(72) Inventor: Glen A. Griffith, Newbury Park, CA (US)

(21) Appl. No.: 18/691,000

(22) PCT Filed: Sep. 28, 2021
(86) PCT No.: PCT/US2021/052433

§ 371 Date: Mar. 11, 2024

(51) Int. Cl. A61N 1/36; A61N 1/372

(52) U.S. Cl. CPC A61N 1/36038; A61N 1/37229; A61N 1/37235

(57) ABSTRACT

An exemplary headpiece includes a headpiece coil comprising a plurality of windings configured to produce a combined output signal that is transcutaneously transmitted to an implant coil of a cochlear implant, the combined output signal configured to control an operation of the cochlear implant. The headpiece further includes a plurality of drivers, each driver of the plurality of drivers coupled to a respective winding of the plurality of windings and configured to drive the respective winding to generate the combined output signal.

### BACKGROUND INFORMATION

Cochlear implants are implanted in recipients to provide benefits for medical purposes. For example, cochlear implants may improve or enable hearing in a recipient lacking full hearing capabilities. Cochlear implant systems may include a component configured to communicate with the cochlear implant. Such components may be directed to generate output signals for communicating with the cochlear implant and/or provide power to the cochlear implant. Such components may also be configured to receive signals and/or power from a sound processor. The providing and receiving of signals and/or power may be dependent on a type of connection between components of the cochlear implant systems.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system.

FIG. 2 shows an exemplary configuration of the cochlear implant system of FIG. 1.

FIG. 3 shows another exemplary configuration of the cochlear implant system of FIG. 1.

FIGS. 4-5 illustrate exemplary configurations of a headpiece of a cochlear implant system.

FIG. 6 illustrates an exemplary graph of an output of a headpiece of a cochlear implant system.

FIG. 7 illustrates an exemplary method to direct a headpiece of a cochlear implant system.

### DETAILED DESCRIPTION

Headpiece configurations of a cochlear implant system are described herein. For example, a headpiece may include a headpiece coil including a plurality of windings configured to produce a combined output signal that is transcutaneously transmitted to an implant coil of a cochlear implant. The combined output signal may be configured to control an operation of the cochlear implant. The headpiece may further include a plurality of drivers, each driver of the plurality of drivers coupled to a respective winding of the plurality of windings and configured to drive the respective winding to generate the combined output signal.

The systems and methods described herein may allow for efficient use of resources in a cochlear implant system. Cochlear implant systems described herein may include a headpiece that includes circuits specific to cochlear implants that were conventionally located in a separate sound processor. As described herein, the headpiece may include a plurality of drivers, with each winding of the headpiece coil driven independently of the other windings. Such configurations may allow for fewer components across the headpiece and sound processor than a conventional cochlear implant system, as conventional headpieces include transformers to modulate power received from the external sound processor. Additionally, configurations described herein may allow the headpiece to be compatible with non-cochlear implant specific sound processors, such as conventional behind-the-ear (BTE) units. Additionally, such configurations may allow efficient power usage, extending battering operating time of the cochlear implant system.

These and other advantages and benefits of the present systems and methods are described in more detail herein.

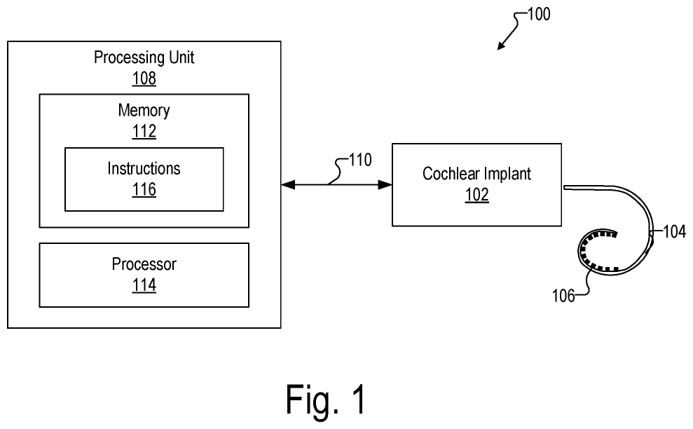

FIG. 1 illustrates an exemplary cochlear implant system 100 configured to be used by a recipient. As shown, cochlear implant system 100 includes a cochlear implant 102, an electrode lead 104 physically coupled to cochlear implant 102 and having an array of electrodes 106, and a processing unit 108 configured to be communicatively coupled to cochlear implant 102 by way of a communication link 110.

The cochlear implant system 100 shown in FIG. 1 is unilateral (i.e., associated with only one ear of the recipient). Alternatively, a bilateral configuration of cochlear implant system 100 may include separate cochlear implants and electrode leads for each ear of the recipient. In the bilateral configuration, processing unit 108 may be implemented by a single processing unit configured to interface with both cochlear implants or by two separate processing units each configured to interface with a different one of the cochlear implants.

Cochlear implant 102 may be implemented by any suitable type of implantable stimulator configured to apply electrical stimulation to one or more stimulation sites located along an auditory pathway of the recipient. In some examples, cochlear implant 102 may additionally or alternatively apply nonelectrical stimulation (e.g., mechanical and/or optical stimulation) to the auditory pathway of the recipient.

In some examples, cochlear implant 102 may be configured to generate electrical stimulation representative of an audio signal processed by processing unit 108 in accordance with one or more stimulation parameters transmitted to cochlear implant 102 by processing unit 108. Cochlear implant 102 may be further configured to apply the electrical stimulation to one or more stimulation sites (e.g., one or more intracochlear locations) within the recipient by way of one or more electrodes 106 on electrode lead 104. In some examples, cochlear implant 102 may include a plurality of independent current sources each associated with a channel defined by one or more of electrodes 106. In this manner, different stimulation current levels may be applied to multiple stimulation sites simultaneously by way of multiple electrodes 106.

Cochlear implant 102 may additionally or alternatively be configured to generate, store, and/or transmit data. For example, cochlear implant 102 may use one or more electrodes 106 to record one or more signals (e.g., one or more voltages, impedances, evoked responses within the recipient, and/or other measurements) and transmit, by way of communication link 110, data representative of the one or more signals to processing unit 108. In some examples, this data is referred to as back telemetry data.

Electrode lead 104 may be implemented in any suitable manner. For example, a distal portion of electrode lead 104 may be pre-curved such that electrode lead 104 conforms with the helical shape of the cochlea after being implanted. Electrode lead 104 may alternatively be naturally straight or of any other suitable configuration.

In some examples, electrode lead 104 includes a plurality of wires (e.g., within an outer sheath) that conductively couple electrodes 106 to one or more current sources within cochlear implant 102. For example, if there are n electrodes 106 on electrode lead 104 and n current sources within cochlear implant 102, there may be n separate wires within electrode lead 104 that are configured to conductively connect each electrode 106 to a different one of the n current sources. Exemplary values for n are 8, 12, 16, or any other suitable number.

Electrodes 106 are located on at least a distal portion of electrode lead 104. In this configuration, after the distal portion of electrode lead 104 is inserted into the cochlea, electrical stimulation may be applied by way of one or more of electrodes 106 to one or more intracochlear locations. One or more other electrodes (e.g., including a ground electrode, not explicitly shown) may also be disposed on other parts of electrode lead 104 (e.g., on a proximal portion of electrode lead 104) to, for example, provide a current return path for stimulation current applied by electrodes 106 and to remain external to the cochlea after the distal portion of electrode lead 104 is inserted into the cochlea. Additionally or alternatively, a housing of cochlear implant 102 may serve as a ground electrode for stimulation current applied by electrodes 106.

Processing unit 108 may be configured to interface with (e.g., control and/or receive data from) cochlear implant 102. For example, processing unit 108 may transmit commands (e.g., stimulation parameters and/or other types of operating parameters in the form of data words included in a forward telemetry sequence) to cochlear implant 102 by way of communication link 110. Processing unit 108 may additionally or alternatively provide operating power to cochlear implant 102 by transmitting one or more power signals to cochlear implant 102 by way of communication link 110. Processing unit 108 may additionally or alternatively receive data from cochlear implant 102 by way of communication link 110. Communication link 110 may be implemented by any suitable number of wired and/or wireless bidirectional and/or unidirectional links.

As shown, processing unit 108 includes a memory 112 and a processor 114 configured to be selectively and communicatively coupled to one another. In some examples, memory 112 and processor 114 may be distributed between multiple devices and/or multiple locations as may serve a particular implementation.

Memory 112 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (RAM), and an optical disc. Exemplary volatile storage media include, but are not limited to, RAM (e.g., dynamic RAM).

Memory 112 may maintain (e.g., store) executable data used by processor 114 to perform one or more of the operations described herein. For example, memory 112 may store instructions 116 that may be executed by processor 114 to perform any of the operations described herein. Instructions 116 may be implemented by any suitable application, program (e.g., sound processing program), software, code, and/or other executable data instance. Memory 112 may also maintain any data received, generated, managed, used, and/or transmitted by processor 114.

Processor 114 may be configured to perform (e.g., execute instructions 116 stored in memory 112 to perform) various operations with respect to cochlear implant 102.

To illustrate, processor 114 may be configured to control an operation of cochlear implant 102. For example, processor 114 may receive an audio signal (e.g., by way of a microphone communicatively coupled to processing unit 108, a wireless interface (e.g., a Bluetooth interface), and/or a wired interface (e.g., an auxiliary input port)). Processor 114 may process the audio signal in accordance with a sound processing program (e.g., a sound processing program stored in memory 112) to generate appropriate stimulation parameters. Processor 114 may then transmit the stimulation parameters to cochlear implant 102 to direct cochlear implant 102 to apply electrical stimulation representative of the audio signal to the recipient.

In some implementations, processor 114 may also be configured to apply acoustic stimulation to the recipient. For example, a receiver (also referred to as a loudspeaker) may be optionally coupled to processing unit 108. In this configuration, processor 114 may deliver acoustic stimulation to the recipient by way of the receiver. The acoustic stimulation may be representative of an audio signal (e.g., an amplified version of the audio signal), configured to elicit an evoked response within the recipient, and/or otherwise configured. In configurations in which processor 114 is configured to both deliver acoustic stimulation to the recipient and direct cochlear implant 102 to apply electrical stimulation to the recipient, cochlear implant system 100 may be referred to as a bimodal hearing system and/or any other suitable term.

Processor 114 may be additionally or alternatively configured to receive and process data generated by cochlear implant 102. For example, processor 114 may receive data representative of a signal recorded by cochlear implant 102 using one or more electrodes 106 and, based on the data, adjust one or more operating parameters of processing unit 108. Additionally or alternatively, processor 114 may use the data to perform one or more diagnostic operations with respect to cochlear implant 102 and/or the recipient.

Other operations may be performed by processor 114 as may serve a particular implementation. In the description provided herein, any references to operations performed by processing unit 108 and/or any implementation thereof may be understood to be performed by processor 114 based on instructions 116 stored in memory 112.

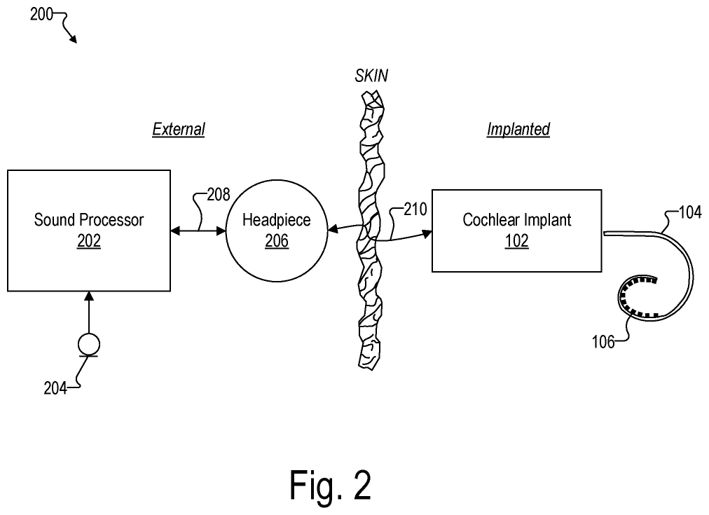

Processing unit 108 may be implemented by one or more devices configured to interface with cochlear implant 102. To illustrate, FIG. 2 shows an exemplary configuration 200 of cochlear implant system 100 in which processing unit 108 is implemented by a sound processor 202 configured to be located external to the recipient. In configuration 200, sound processor 202 is communicatively coupled to a microphone 204 and to a headpiece 206 that are both configured to be located external to the recipient.

Sound processor 202 may be implemented by any suitable device that may be worn or carried by the recipient. For example, sound processor 202 may be implemented by a behind-the-ear (BTE) unit configured to be worn behind and/or on top of an ear of the recipient. Additionally or alternatively, sound processor 202 may be implemented by an off-the-ear unit (also referred to as a body worn device) configured to be worn or carried by the recipient away from the ear. Additionally or alternatively, at least a portion of sound processor 202 is implemented by circuitry within headpiece 206.

Microphone 204 is configured to detect one or more audio signals (e.g., that include speech and/or any other type of sound) in an environment of the recipient. Microphone 204 may be implemented in any suitable manner. For example, microphone 204 may be implemented by a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 202. Additionally or alternatively, microphone 204 may be implemented by one or more microphones in or on headpiece 206, one or more microphones in or on a housing of sound processor 202, one or more beam-forming microphones, and/or any other suitable microphone as may serve a particular implementation.

Headpiece 206 may be selectively and communicatively coupled to sound processor 202 by way of a communication link 208 (e.g., a cable or any other suitable wired or wireless communication link), which may be implemented in any suitable manner. Headpiece 206 may include an external antenna (e.g., a coil and/or one or more wireless communication components) configured to facilitate selective wireless coupling of sound processor 202 to cochlear implant 102. Headpiece 206 may additionally or alternatively be used to selectively and wirelessly couple any other external device to cochlear implant 102. To this end, headpiece 206 may be configured to be affixed to the recipient's head and positioned such that the external antenna housed within headpiece 206 is communicatively coupled to a corresponding implantable antenna (which may also be implemented by a coil and/or one or more wireless communication components) included within or otherwise connected to cochlear implant 102. In this manner, stimulation parameters and/or power signals may be wirelessly and transcutaneously transmitted between sound processor 202 and cochlear implant 102 by way of a wireless communication link 210.

In configuration 200, sound processor 202 may receive an audio signal detected by microphone 204 by receiving a signal (e.g., an electrical signal) representative of the audio signal from microphone 204. Sound processor 202 may additionally or alternatively receive the audio signal by way of any other suitable interface as described herein. Sound processor 202 may process the audio signal in any of the ways described herein and transmit, by way of headpiece 206, stimulation parameters to cochlear implant 102 to direct cochlear implant 102 to apply electrical stimulation representative of the audio signal to the recipient.

In an alternative configuration, sound processor 202 may be implanted within the recipient instead of being located external to the recipient. In this alternative configuration, which may be referred to as a fully implantable configuration of cochlear implant system 100, sound processor 202 and cochlear implant 102 may be combined into a single device or implemented as separate devices configured to communicate one with another by way of a wired and/or wireless communication link. In a fully implantable implementation of cochlear implant system 100, headpiece 206 may not be included and microphone 204 may be implemented by one or more microphones implanted within the recipient, located within an ear canal of the recipient, and/or external to the recipient.

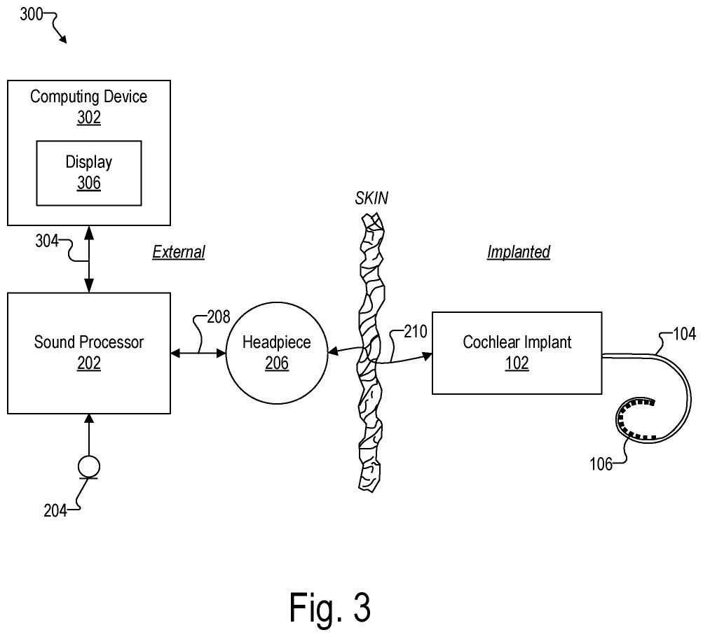

FIG. 3 shows an exemplary configuration 300 of cochlear implant system 100 in which processing unit 108 is implemented by a combination of sound processor 202 and a computing device 302 configured to communicatively couple to sound processor 202 by way of a communication link 304, which may be implemented by any suitable wired or wireless communication link.

Computing device 302 may be implemented by any suitable combination of hardware and software. To illustrate, computing device 302 may be implemented by a mobile device (e.g., a mobile phone, a laptop, a tablet computer, etc.), a desktop computer, and/or any other suitable computing device as may serve a particular implementation. As an example, computing device 302 may be implemented by a mobile device configured to execute an application (e.g., a mobile app) that may be used by a user (e.g., the recipient, a clinician, and/or any other user) to control one or more settings of sound processor 202 and/or cochlear implant 102 and/or perform one or more operations (e.g., diagnostic operations) with respect to data generated by sound processor 202 and/or cochlear implant 102.

In some examples, computing device 302 may be configured to control an operation of cochlear implant 102 by transmitting one or more commands to cochlear implant 102 by way of sound processor 202. Likewise, computing device 302 may be configured to receive data generated by cochlear implant 102 by way of sound processor 202. Alternatively, computing device 302 may interface with (e.g., control and/or receive data from) cochlear implant 102 directly by way of a wireless communication link between computing device 302 and cochlear implant 102. In some implementations in which computing device 302 interfaces directly with cochlear implant 102, sound processor 202 may or may not be included in cochlear implant system 100.

Computing device 302 is shown as having an integrated display 306. Display 306 may be implemented by a display screen, for example, and may be configured to display content generated by computing device 302. Additionally or alternatively, computing device 302 may be communicatively coupled to an external display device (not shown) configured to display the content generated by computing device 302.

In some examples, computing device 302 represents a fitting device configured to be selectively used (e.g., by a clinician) to fit sound processor 202 and/or cochlear implant 102 to the recipient. In these examples, computing device 302 may be configured to execute a fitting program configured to set one or more operating parameters of sound processor 202 and/or cochlear implant 102 to values that are optimized for the recipient. As such, in these examples, computing device 302 may not be considered to be part of cochlear implant system 100. Instead, computing device 302 may be considered to be separate from cochlear implant system 100 such that computing device 302 may be selectively coupled to cochlear implant system 100 when it is desired to fit sound processor 202 and/or cochlear implant 102 to the recipient.

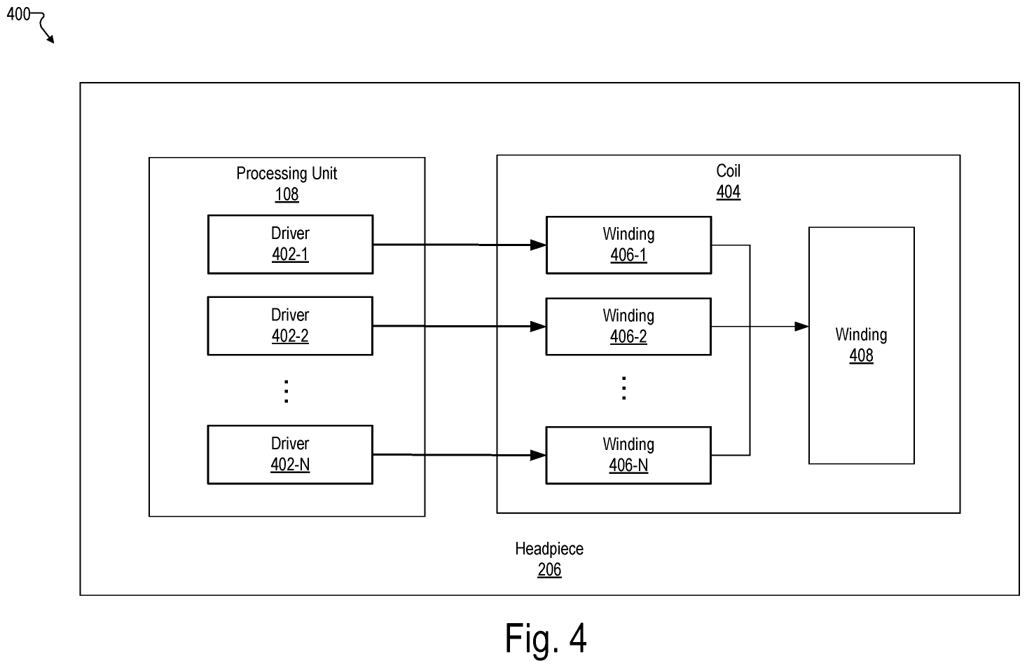

FIG. 4 illustrates an exemplary configuration 400 of a headpiece (e.g., headpiece 206) of a cochlear implant system (e.g., cochlear implant system 100). As shown, in configuration 400, headpiece 206 includes a plurality of drivers 402 (e.g., drivers 402-1 through 402-N) configured to drive (e.g., control by selectively providing power to) a coil 404. In some examples, one or more of drivers 402 may be included in and/or implemented by a processing unit (e.g., processing unit 108) of headpiece 206. Coil 404 may be configured to transcutaneously transmit signals to a cochlear implant (e.g., cochlear implant 102), such as power signals and/or stimulation parameters for cochlear implant 102 to represent audio signals to a recipient. Coil 404 may include a plurality of windings 406 (e.g., windings 406-1 through 406-N) and an output winding 408. Each winding 406 (of the plurality of windings 406) may be coupled to a respective driver 402. Consequently, each driver 402 may drive each winding 406 independently, such that a combined output of windings 406 may correspond to the signals that are transmitted to cochlear implant 402. The combined output may be transmitted via an output winding 408 of coil 404.

Drivers 402 may be implemented by any suitable circuit. For instance, drivers 402 may be implemented as complementary class D amplifiers, each including a complementary metal-oxide-semiconductor (CMOS) logic inverter including a p-type metal-oxide-semiconductor (PMOS) and n-type metal-oxide-semiconductor (NMOS) transistor. One transistor at a time may be active, either connecting an output of driver 402 to a voltage supply or to a negative return. Additionally or alternatively, any other suitable driver circuit may be used.

Each pair of driver 402 with winding 406 may be configured to generate a different output signal so that a combination of the outputs of each pair may produce a signal for controlling cochlear implant 102. For instance, each winding 406 may have a particular number of turns to establish a particular input impedance and, consequently, a ratio of currents for windings 406. Additionally or alternatively, each pair may be further coupled to a respective resonating capacitor, with each resonating capacitor having a particular capacitance that may be used to establish a ratio of currents through each winding 406.

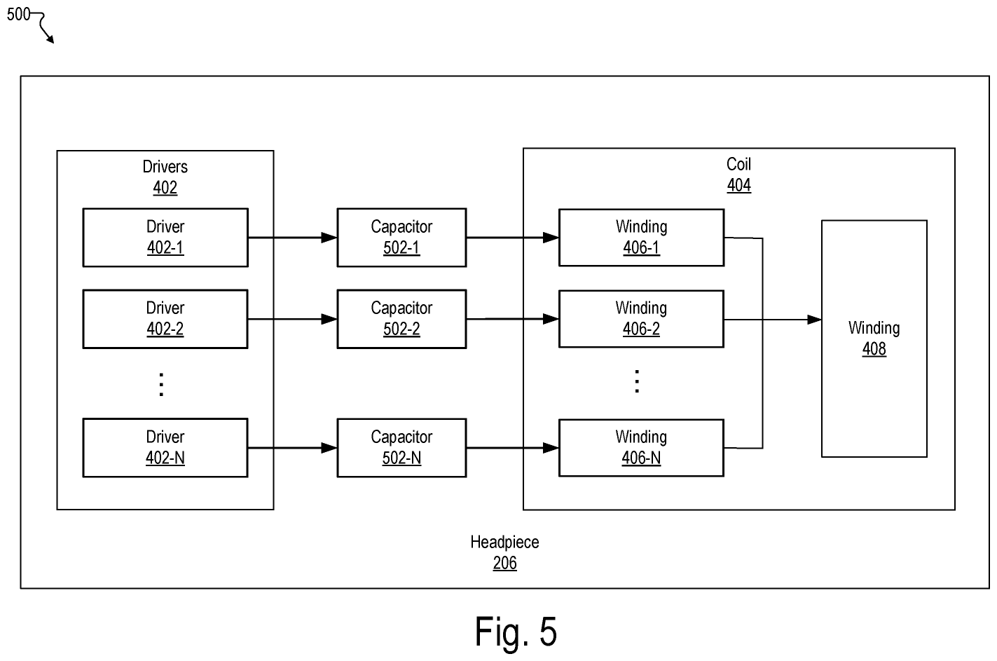

FIG. 5 shows an example configuration 500 of headpiece 206 that includes capacitors 502. Configuration 500 may be similar to configuration 400, including drivers 402 and a coil 404 that includes windings 406 and an output winding 408. In some examples, drivers 402 may be implemented separate from processing unit 108. As shown, each pair of driver 402 with winding 406 is further coupled to a respective capacitor 502.

Capacitors 502 may allow for non-integer ratios between driver 402-winding 406 pairs, which may allow for finer control of the outputs of each winding 406 and/or a higher quality (Q) factor than relying on just the ratio of turns of windings 406. In some examples, capacitors 502 may further allow for windings 406 to be implemented using a same ratio of turns, which may allow for simplification of components.

In some examples, the configurations of each driver 402-winding 406 pair may allow for drivers 402 to be coupled to a single fixed voltage supply (e.g., a battery) without a voltage regulator. In other examples, one or more of drivers 402 may be coupled to different voltage supplies, a variable voltage supply, and/or a voltage regulator.

While configurations 400 and 500 show each winding 406 coupled to a different driver 402, in some examples, one or more additional windings may contribute to the combined output, such as windings coupled to a same driver 402 as another winding, etc.

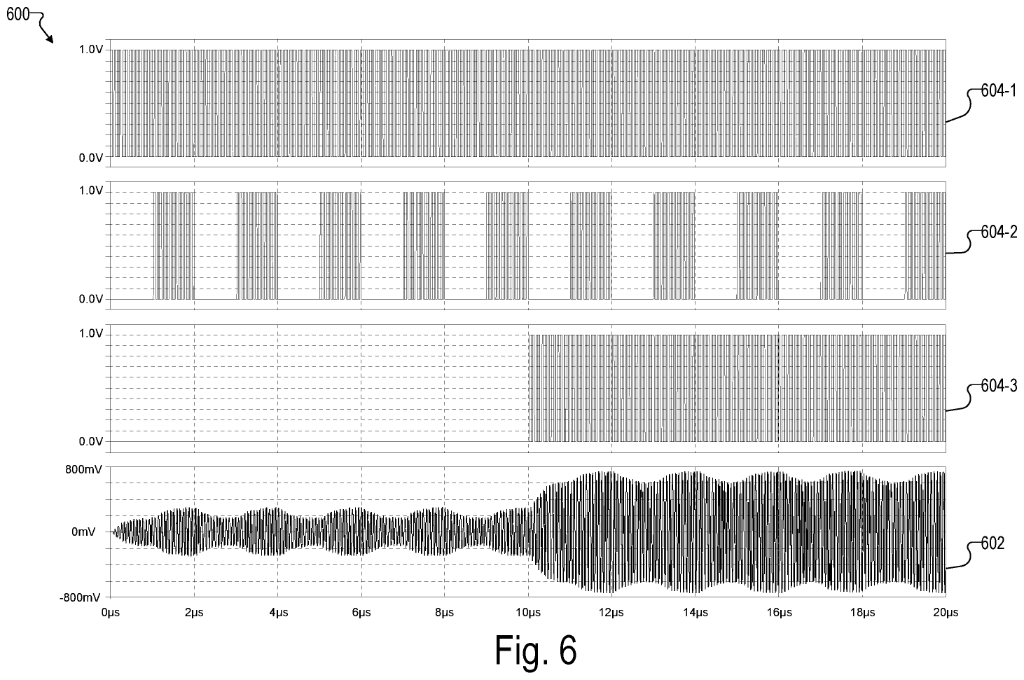

FIG. 6 illustrates an exemplary graph 600 showing a combined output signal 602 configured to control an operation of a cochlear implant (e.g., cochlear implant 102). Combined output signal 602 may be output by an output winding (e.g., output winding 408) of a coil (e.g., coil 404) of a headpiece (e.g., headpiece 206). Combined output signal 602 may be generated as a combination of outputs 604 from a plurality of windings (e.g., windings 406), each driven by a respective driver (e.g., drivers 402). In this example, the plurality of windings 406 may include three windings 406 driven (e.g., controlled) by three drivers 402, respectively. The three outputs 604 may implement amplitude-shift keying modulation to generate combined output signal 602.

Output 604-1 may be generated by a first winding 406-1 controlled by a first driver 402-1. Driver 402-1 may be implemented as a carrier generator that may run continuously while headpiece 206 is transmitting and provide a majority of the power. The corresponding output 604-1 may be a carrier signal that provides a baseline for combined output signal 602. Output 604-2 may be generated by a second winding 406-2 controlled by a second driver 402-2. Driver 402-2 may be implemented as a behavioral voltage generator. Driver 402-2 may use a same carrier signal as driver 402-1 but may be keyed on and off to encode a data sequence to be transmitted through combined output signal 602. As shown, output 604-2 has a same magnitude as the carrier signal output 604-1, but may be turned on and off to implement on-off keying. Output 604-3 may be generated by a third winding 406-3 controlled by a third driver 402-3. Driver 402-3 may be implemented as a boost driver, which, when active, may be configured to increase a magnetic flux and a magnitude of combined output signal 602.

Graph 600 shows outputs 604 combining to generate combined output signal 602. As shown, combined output signal 602 has a baseline based on carrier signal output 604-1, keyed by data sequence signal output 604-2. Combined output signal 602 is additionally magnified by boost signal output 604-3. Combined output signal 602 may be output by output winding 408 to control cochlear implant 102, such as to generate and apply electrical stimulation in a particular pattern to represent an audio signal to a recipient of cochlear implant 102. The audio signal may be any suitable audio signal, such as an audio signal received by headpiece 206 by way of a sound processor (e.g., sound processor 202). As described, sound processor 202 may be implemented as a component separate from (e.g., not included in) headpiece 206 and/or by components included in headpiece 206.

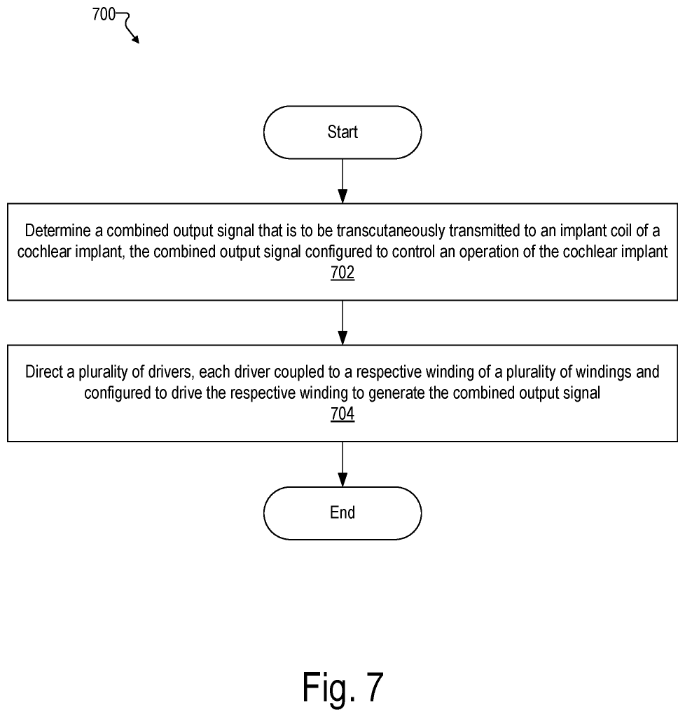

FIG. 7 illustrates an exemplary method 700. The operations shown in FIG. 7 may be performed by headpiece 206, a processing unit of headpiece 206, processing unit 108 and/or any implementation thereof. While FIG. 7 illustrates exemplary operations according to one embodiment, other embodiments may omit, add to, reorder, and/or modify any of the operations shown in FIG. 7.

At operation 702, a headpiece of a cochlear implant system determines a combined output signal that is to be transcutaneously transmitted to an implant coil of the cochlear implant, the combined output signal configured to control an operation of the cochlear implant. Operation 702 may be performed in any of the ways described herein.

At operation 704, the headpiece directs a plurality of drivers, each driver coupled to a respective winding of a plurality of windings and configured to drive the respective winding to generate the combined output signal. Operation 704 may be performed in any of the ways described herein.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A headpiece for use in a cochlear implant system comprising:
a headpiece coil comprising a plurality of windings configured to produce a combined output signal that is transcutaneously transmitted to an implant coil of the cochlear implant, the combined output signal configured to control an operation of the cochlear implant; and
a plurality of drivers, each driver of the plurality of drivers coupled to a respective winding of the plurality of windings and configured to drive the respective winding to generate the combined output signal.

2. The headpiece of claim 1, wherein the plurality of drivers are configured to drive the plurality of windings to generate the combined output using amplitude-shift keying (ASK) modulation.

3. The headpiece of claim 2, wherein:
a first driver of the plurality of drivers is configured to drive a first winding of the plurality of windings to generate a carrier signal that provides a baseline for the combined output signal; and
a second driver of the plurality of drivers is configured to drive a second winding of the plurality of windings to generate, using on-off keying, a data sequence signal that encodes a data sequence for the combined output signal.

4. The headpiece of claim 3, wherein a third driver of the plurality of drivers is configured to drive a third winding of the plurality of windings to generate a boost signal that amplifies the combined output signal.

5. The headpiece of claim 1, further comprising a plurality of capacitors, each capacitor of the plurality of capacitors coupled to a respective winding included in the plurality of windings and configured to generate a non-integer ratio between respective outputs of the plurality of windings.

6. The headpiece of claim 1, wherein the combined output signal is configured to control the operation of the cochlear implant by directing the cochlear implant to generate and apply electrical stimulation representative of an audio signal to a recipient.

7. The headpiece of claim 1, wherein the combined output signal is generated based on a signal received by the headpiece by way of a sound processor not included in the headpiece.

8. The headpiece of claim 7, wherein the sound processor is implemented by a behind-the-ear unit configured to be worn by a recipient.

9. The headpiece of claim 1, wherein the headpiece is configured to be worn on a head of a recipient.

10. A cochlear implant system comprising:
a cochlear implant configured to be implanted within a recipient; and
a headpiece comprising:
a headpiece coil comprising a plurality of windings configured to produce a combined output signal that is transcutaneously transmitted to an implant coil of the cochlear implant, the combined output signal configured to control an operation of the cochlear implant; and
a plurality of drivers, each driver of the plurality of drivers coupled to a respective winding of the plurality of windings and configured to drive the respective winding to generate the combined output signal.

11. The cochlear implant system of claim 10, wherein the plurality of drivers are configured to drive the plurality of windings to generate the combined output using amplitude-shift keying (ASK) modulation.

12. The cochlear implant system of claim 11, wherein:
a first driver of the plurality of drivers is configured to drive a first winding of the plurality of windings to generate a carrier signal that provides a baseline for the combined output signal; and
a second driver of the plurality of drivers is configured to drive a second winding of the plurality of windings to generate, using on-off keying, a data sequence signal that encodes a data sequence for the combined output signal.

13. The cochlear implant system of claim 12, wherein a third driver of the plurality of drivers is configured to drive a third winding of the plurality of windings to generate a boost signal that amplifies the combined output signal.

14. The cochlear implant system of claim 10, further comprising a plurality of capacitors, each capacitor of the plurality of capacitors coupled to a respective winding included in the plurality of windings and configured to generate a non-integer ratio between respective outputs of the plurality of windings.

15. The cochlear implant system of claim 10, wherein the combined output signal is configured to control the operation of the cochlear implant by directing the cochlear implant to generate and apply electrical stimulation representative of an audio signal to the recipient.

16. The cochlear implant system of claim 10, further comprising a behind-the-ear unit configured to be worn by the recipient and detect an audio signal to the recipient.

17. The cochlear implant system of claim 10, wherein the headpiece is configured to be worn on a head of the recipient.

18. A method comprising:
determining, by a processing unit of a cochlear implant system, a combined output signal that is to be transcutaneously transmitted to an implant coil of the cochlear implant, the combined output signal configured to control an operation of the cochlear implant;
directing, by the processing unit, a plurality of drivers, each driver coupled to a respective winding of a plurality of windings and configured to drive the respective winding to generate the combined output signal.

19. The method of claim 18, wherein the directing the plurality of drivers comprises directing the drivers to drive the plurality of windings to generate the combined output using amplitude-shift keying (ASK) modulation.

20. The method of claim 18, wherein the combined output signal is configured to control the operation of the cochlear implant by directing the cochlear implant to generate and apply electrical stimulation representative of an audio signal to a recipient.
